(19) United States
(12) Patent Application Publication
(10) Pub. No.: US 2011/0238108 A1
(43) Pub. Date: Sep. 29, 2011

(54) Surgical instrument

(76) Inventors: William J. Peine, Ashland, MA (US); Andres Chamorro, Waltham, MA (US); Woojin Lee, Hopkinton, MA (US)

(21) Appl. No.: 12/661,765

(22) Filed: Mar. 23, 2010

(51) Int. Cl. A61B 17/00

(52) U.S. Cl. 606/205

(57) ABSTRACT

A surgical instrument that includes an instrument shaft having proximal and distal ends; a tool disposed from the distal end of the instrument shaft; a control handle coupled from the proximal end of the instrument shaft; a distal bendable member for coupling the distal end of the instrument shaft to the tool; a proximal bendable member for coupling the proximal end of the instrument shaft to the control handle; actuation means extending between the distal and proximal bendable members for coupling motion of the proximal bendable member to the distal bendable member for controlling the positioning of the tool and a locking mechanism for fixing the position of the tool at a selected position. The locking mechanism includes a ball and socket arrangement disposed about the proximal bendable member and a locking member for locking the ball and socket arrangement and having locked and unlocked states. The ball and socket arrangement includes a compression ring supported from the control handle, having an outer surface for support of the locking member thereabout and having an inner surface defining an at least partially spherical shaped socket. The ball and socket arrangement further includes a hollow ball member having an internal hollow chamber and an outer at least partially spherical shaped surface which mates with the at least partially spherical shaped socket.

### TECHNICAL FIELD

The present invention relates in general to medical instruments, and more particularly to manually-operated surgical instruments that are intended for use in minimally invasive surgery or other forms of surgical or medical procedures or techniques. The instrument described herein is primarily for a laparoscopic procedure, however, it is to be understood that the instrument of the present invention can be used for a wide variety of other procedures, including intraluminal procedures.

### BACKGROUND OF THE INVENTION

Endoscopic and laparoscopic instruments currently available in the market are extremely difficult to learn to operate and use, mainly due to a lack of dexterity in their use. For instance, when using a typical laparoscopic instrument during surgery, the orientation of the tool of the instrument is solely dictated by the location of the target and the incision. These instruments generally function with a fulcrum effect using the patient own incision area as the fulcrum. As a result, common tasks such as suturing, knotting and fine dissection have become challenging to master. Various laparoscopic instruments have been developed over the years to overcome this deficiency, usually by providing an extra articulation often controlled by a separately disposed control member for added control. However, even so these instruments still do not provide enough dexterity to allow the surgeon to perform common tasks such as suturing, particularly at any arbitrarily selected orientation.

The goal of minimally invasive surgery (MIS) is to manipulate tissues within the human body while minimizing damage to the surrounding healthy organs. Laparoscopy, for example, uses endoscopic cameras and long slender instruments to perform surgery through a few small (1-2 cm) skin incisions. This provides many benefits to patients over traditional open incision techniques, including fewer infections, less pain, shorter hospital stays, faster recovery times, and less scaring. These advantages have allowed surgeons to apply MIS to procedures in every surgical specialty. During the 1990s, the growth rate of MIS was tremendous; however, in the last few years the application to new procedures has largely stalled due to limitations in visualization, access, and control. It is a general belief among surgeons that a new wave of technology is needed in order for MIS to reach the next level. Smaller cameras and instruments that can flexibly navigate around organs with added dexterity will allow them to perform surgery not possible today.

Prior laparoscopic and endoscopic instruments were a simple adaptation of tools used for open incision surgery. They are similar in mechanical construction with the addition of a long, 2˜10 mm diameter shaft between the handle and end effectors. They lack the dexterity of open incision surgery due to the ulcrum effect Since the tools pivot about the incision, they are generally limited to 5 Degrees-of-Freedom (DOF): pivoting up/down, pivoting left/right, sliding in/out, rotating about the shaft axis, and actuation of the jaws. In contrast, open incision surgery allows full dexterity (7 DOF) due to the surgeon wrist, with additional DOF from their elbow and shoulder used to avoid obstacles and optimize access to the tissue. Further complicating MIS, the surgeon views the operative site on a monitor located outside the sterile field. This displacement between eyes and hands combined with the reversal of motions caused from the fulcrum effect makes these techniques difficult to learn and master. It takes the skills of an experienced surgeon to consistently perform advanced MIS at a high level.

Surgery now in virtually every surgical discipline is moving toward making MIS more minimal. This means using smaller and fewer incisions, or most ideally, no incisions. The art has already made the transition from open to endoscopic surgery; now surgeons are pioneering surgical techniques that use the patient natural orifices as entry points into the body. These approaches further reduce pain and recovery times and, in many cases, produce no visible scars. One fairly new technique is referred to as single port access surgery (or SPA). This is a type of laparoscopy where all the instruments and laparoscope enter the abdominal cavity through one incision. Most of these procedures use the umbilicus for the entry port location because it heals quickly, does not have significant muscle groups below it, and hides any scaring well.

An improved instrument is shown in U.S. Pat. No. 7,147,650 having enhanced dexterity and including, inter alia, a rotation feature with proximal and distal bendable members. Other instruments are also shown in U.S. Pat. No. 7,364,582 and U.S. Pat. No. 7,338,513,commonly owned by the assignee of the present invention. Even though these instruments have improved features there remains the need for a more economically feasible instrument and, in particular, an instrument in which the handle can be re-used while the tip of the instrument is disposable or reposable.

Reference is now also made to two other applications co-pending and co-owned with the present invention and identified as published applications 2009/0069842 and 2009/0171147. These applications illustrate various instrument constructions some of which include a disposable or reposable instrument structure.

Accordingly, an object of the present invention is to provide an improved laparoscopic or endoscopic instrument in which a portion of the instrument is re-useable and a portion is disposable or reposable. In embodiments described herein the handle end of the instrument is re-useable and the distal portion or tip of the instrument is disposable or reposable. By being able to re-use the handle portion, the instrument is more economically feasible.

Still another object of the present invention is to provide an improved laparoscopic or endoscopic instrument in which a portion of the instrument is re-useable and a portion is disposable or reposable. By constructing the instrument in this manner this allows the ready substitution of various end effectors useful in performing a surgical procedure.

Still a further object of the present invention is to provide a resposable instrument construction in which the substitutable shaft portion is engaged in a rear entry or rear load manner.

A further object of the present invention is to provide an improved laparoscopic or endoscopic surgical instrument that allows the surgeon to manipulate the tool end of the surgical instrument with greater dexterity.

Another object of the present invention is to provide an improved surgical or medical instrument that has a wide variety of applications, through incisions, through natural body orifices or intraluminally.

Another object of the present invention is to provide a locking feature that is an important adjunct to the other controls of the instrument enabling the surgeon to lock the instrument once in the desired position. This makes it easier for the surgeon to thereafter perform surgical procedures without having to, at the same time, hold the instrument in a particular bent configuration.

Still another object of the present invention is to provide an improved medical instrument that can be effectively controlled with a single hand of the user.

### SUMMARY OF THE INVENTION

To accomplish the foregoing and other advantages and features of the present invention there is provided a surgical instrument comprising: an instrument shaft having proximal and distal ends; a tool disposed from the distal end of the instrument shaft; a control handle coupled from the proximal end of the instrument shaft; a distal bendable member for coupling the distal end of the instrument shaft to the tool; a proximal bendable member for coupling the proximal end of the instrument shaft to the control handle; actuation means extending between distal and proximal bendable members for coupling motion of the proximal bendable member to the distal bendable member for controlling the positioning of the tool and a locking mechanism for fixing the position of the tool at a selected position. The locking mechanism includes a ball and socket arrangement disposed about said proximal bendable member and a locking member for locking the ball and socket arrangement and having locked and unlocked states. The ball and socket arrangement includes a compression ring supported from the control handle, having an outer surface for support of the locking member thereabout and having an inner surface defining an at least partially spherical shaped socket. The ball and socket arrangement further includes a hollow ball member having an internal hollow chamber and an outer at least partially spherical shaped surface which mates with the at least partially spherical shaped socket.

In accordance with other aspects of the present invention the hollow ball member may be supported within the socket and constructed and arranged with at least a portion of the proximal bendable member disposed in the internal hollow chamber of the hollow ball member; the compression ring may include an annular resilient base member, and a plurality of stiffener segments that are disposed about the annular resilient base member; the annular resilient base member may include a plurality of spacedly disposed hinges that define therebetween a plurality of support segments; the plurality of stiffener segments may be disposed over the respective plurality of support segments; each stiffener segment preferably has, on a top surface thereof, a series of ribs, wherein the ribs are spaced apart and extend circumferentially; including at least a hub for supporting the compression ring; including a plurality of struts for supporting the hub from the control handle; wherein each hinge includes a passage for receiving a respective strut; including a rotation means disposed adjacent the control handle and rotatable relative to the control handle for causing a correspond may comprise a rotation knob that is adapted to rotate the tool about a distal tool roll axis and said rotation knob is disposed between said control handle and proximal bendable member; wherein the control handle may comprise a pistol grip handle having an engagement horn to assist in holding the handle; including an actuation lever supported from the pistol grip handle and a multiple linkage mechanism for supporting the actuation lever; and including a tool actuation cable that extends from the tool to the handle, a slider for capturing the proximal end of the tool actuation cable and an actuation lever supported at the handle for controlling the translation of the slider.

In accordance with other aspects and features of the present invention there is provided a medical instrument comprising a proximal control handle and a distal tool that are intercoupled by an elongated instrument shaft that is meant to pass internally of an anatomic body, proximal and distal bendable members that respectively intercouple the proximal control handle and the distal tool with the instrument shaft, cable actuation means disposed between said bendable members, for controlling the positioning of the distal tool from the control handle, and a locking mechanism for fixing the position of the tool at a selected position; the locking mechanism comprising a ball and socket structure coupled between the control handle and proximal bendable member and a locking member for locking the ball and socket structure and having locked and unlocked states; a compressible ring defining a partially spherical shaped socket of the ball and socket structure; a hollow ball member of the ball and socket structure having an internal hollow chamber and an outer partially spherical shaped surface that mates with the partially spherical shaped socket; the locking member including an annular locking ring disposed about the compressible ring, and in the locked state, compressing the socket relative to the ball member so as to fix the position of the proximal bendable member and, in turn, the distal bendable member and tool.

In accordance with another version of the present invention there is provided a surgical instrument comprising: an instrument shaft having proximal and distal ends; a tool disposed from the distal end of the instrument shaft; a control handle coupled from the proximal end of the instrument shaft; a distal bendable member for coupling the distal end of said instrument shaft to the tool; a proximal bendable member for coupling the proximal end of the instrument shaft to the control handle; actuation means extending between distal and proximal bendable members for coupling motion of the proximal bendable member to the distal bendable member for controlling the positioning of the tool and a tool actuation member that is mounted at and operable from the control handle. The tool actuation member comprises an actuation lever extending from the control handle so as to be operable by a user, and a linkage mechanism supported at the control handle and operable from the actuation lever with the linkage mechanism including a plurality of separate links, one of which is pivotally supported with the actuation lever.

In accordance with still other aspects of the present invention including a tool actuation cable and a slider coupled with the tool actuation cable and controlled from the actuation lever; including a ratchet and pawl mechanism in the handle for allowing a ratcheting action by the actuation lever; including a release button on the handle coupled to the ratchet and pawl mechanism for releasing the ratchet and pawl mechanism; including a slide mechanism on the handle for dis-engaging the ratchet and pawl mechanism; including a pair of parallel disposed links supported between the actuation lever and the handle with one of the links having a ratchet surface forming part of the ratchet and pawl mechanism, a third links pivotally supported in the handle and including a distal pawl forming part of the ratchet and pawl mechanism, and a fourth link coupled between one of the pair of links and the slider; wherein the release button has a spring arm, and the release button is coupled with the third link and including a locking mechanism for fixing the position of the tool at a selected position, the locking mechanism including a ball and socket arrangement disposed about said proximal bendable member and a locking member for locking said ball and socket arrangement and having locked and unlocked states, the ball and socket arrangement including a compression ring supported from the control handle, having an outer surface for support of the locking member thereabout and having an inner surface defining an at least partially spherical shaped socket and the ball and socket arrangement further including a hollow ball member having an internal hollow chamber and an outer at least partially spherical shaped surface that mates with the at least partially spherical shaped socket.

### DESCRIPTION OF THE DRAWINGS

Numerous other objects, features and advantages of the present invention can now be realized in accordance with the present invention by referring to the accompanying drawings, in which:

FIG. 1 is a perspective view of a preferred embodiment of a surgical instrument constructed in accordance with the principles of the present invention;

FIG. 2 is cross-sectional side view as taken along line 2-2 of FIG. 1 and illustrating further details of the instrument;

FIGS. 2A-2C represent a series of cross-sectional views as illustrated in FIG. 2, but illustrating a sequence of positions of the instrument actuation lever;

FIG. 2D is a fragmentary perspective view illustrating details at the instrument handle;

FIG. 3 is a cross-sectional view taken along line 3-3 of FIG. 2;

FIG. 3A is an enlarged cross-sectional detail view taken along line 3A-3A of FIG. 3;

FIG. 4 is a cross-sectional view similar to the one shown in FIG. 3 but with the cinch ring in a released position;

FIG. 4A is an enlarged cross-sectional detail view similar to the one shown in FIG. 3A but with the cinch ring in a released position;

FIG. 5 is a schematic fragmentary cross-sectional view of the angle locking means of FIG. 2 in a bent configuration;

FIG. 6 is an exploded perspective view of the angle locking means, as illustrated in the cross-sectional view of FIG. 5;

FIG. 7 is a perspective view similar to that illustrated in FIG. 1 but of a second embodiment of the surgical instrument, illustrating a needle driver with only a ratcheting action;

FIG. 8 is cross-sectional side view similar to that illustrated FIG. 2 of this second embodiment of surgical instrument with the needle driver tool;

FIG. 9 is a perspective view similar to that illustrated in FIG. 1 but of a third embodiment of the surgical instrument, illustrating a scissors without any ratcheting action;

FIG. 10 is a cross-sectional side view similar to that illustrated in FIG. 2 of this third embodiment of the surgical instrument with a scissors tool;

FIG. 11 is a perspective view similar to that illustrated in FIG. 1 but of a fourth embodiment of the surgical instrument using a cautery tool; and

FIG. 12 is a cross-sectional side view similar to that illustrated in FIG. 2 of this fourth embodiment of the surgical instrument with a cautery tool.

### DETAILED DESCRIPTION

The present invention is illustrated in the drawings as a surgical instrument that is particularly characterized by an improved locking mechanism to maintain the angle locking means in a firmly locked state.

The instrument of the present invention may be used to perform minimally invasive procedures. Minimally invasive procedure, refers herein to a surgical procedure in which a surgeon operates through a small cut or incision, the small incision being used to access the operative site. In one embodiment, the incision length ranges from 1 mm to 20 mm in diameter, preferably from 5 mm to 10 mm in diameter. This procedure contrasts those procedures requiring a large cut to access the operative site. Thus, the flexible instrument is preferably used for insertion through such small incisions and/or through a natural body lumen or cavity, so as to locate the instrument at an internal target site for a particular surgical or medical procedure. The introduction of the surgical instrument into the anatomy may also be by percutaneous or surgical access to a lumen, vessel or cavity, or by introduction through a natural orifice in the anatomy. In addition to use in a laparoscopic procedure, the instrument of the present invention may be used in a variety of other medical or surgical procedures including, but not limited to, colonoscopic, upper GI, arthroscopic, sinus, thorasic, prostate, transvaginal, orthopedic and cardiac procedures. Depending upon the particular procedure, the instrument shaft may be rigid, semi-rigid or flexible.

Although reference is made herein to a surgical instrument, it is contemplated that the principles of this invention also apply to other medical instruments, not necessarily for surgery, and including, but not limited to, such other implements as catheters, as well as diagnostic and therapeutic instruments and implements.

There are a number of unique features embodied in the instrument that is described herein. For example, there is provided a locking mechanism that is constructed using a ball and socket arrangement disposed about the proximal motion member that follows the bending action and in which an annular cinch ring is used to retain the ball and socket arrangement in a fixed particular position, and thus also maintain the proximal and distal bendable members in a particular bent condition, or in other words locked in that position. The cinch ring preferably includes a locking lever that is conveniently located adjacent to the instrument handle and that is easily manipulated to lock and unlock the cinch ring and, in turn, the position of the end effector. The cinch ring is also preferably rotatable to that the locking lever can be positioned conveniently or can be switched (rotated) between left and right handed users. This lock control allows the surgeon one less degree of freedom to concentrate on when performing certain tasks. By locking the bendable sections at a particular position, this enables the surgeon to be more hands-free for controlling other degrees of freedom of the instrument such as manipulation of the rotation knob to, in turn, control the orientation of the end effector.

A main feature of the present invention relates to the aforementioned locking mechanism and the ability of the locking mechanism to have an improved construction so as to provide a more firm locking of the angled position of the angle locking mechanism.

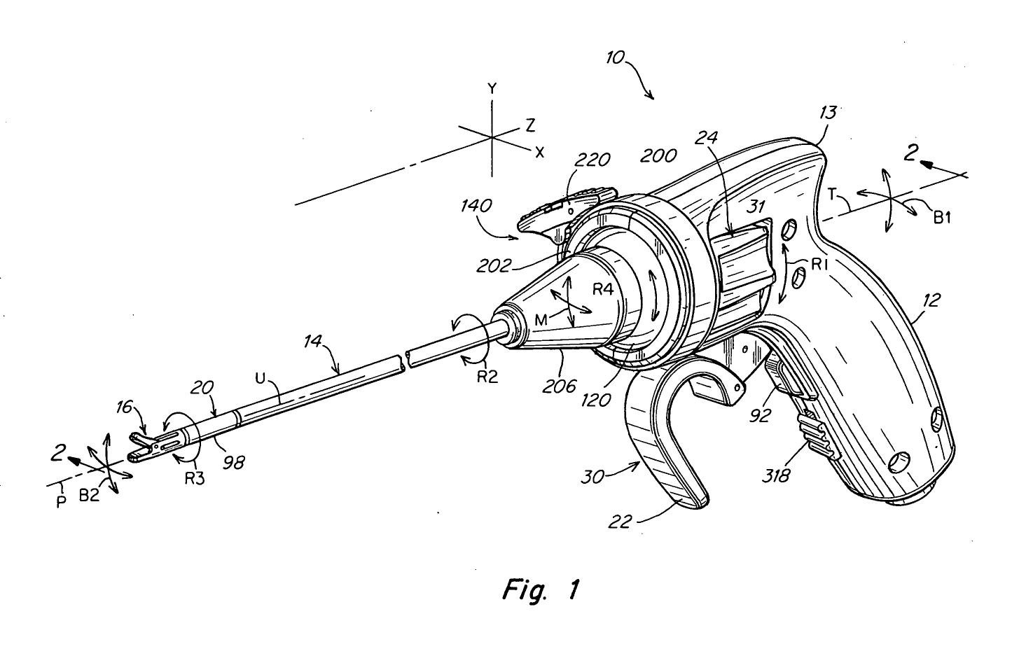

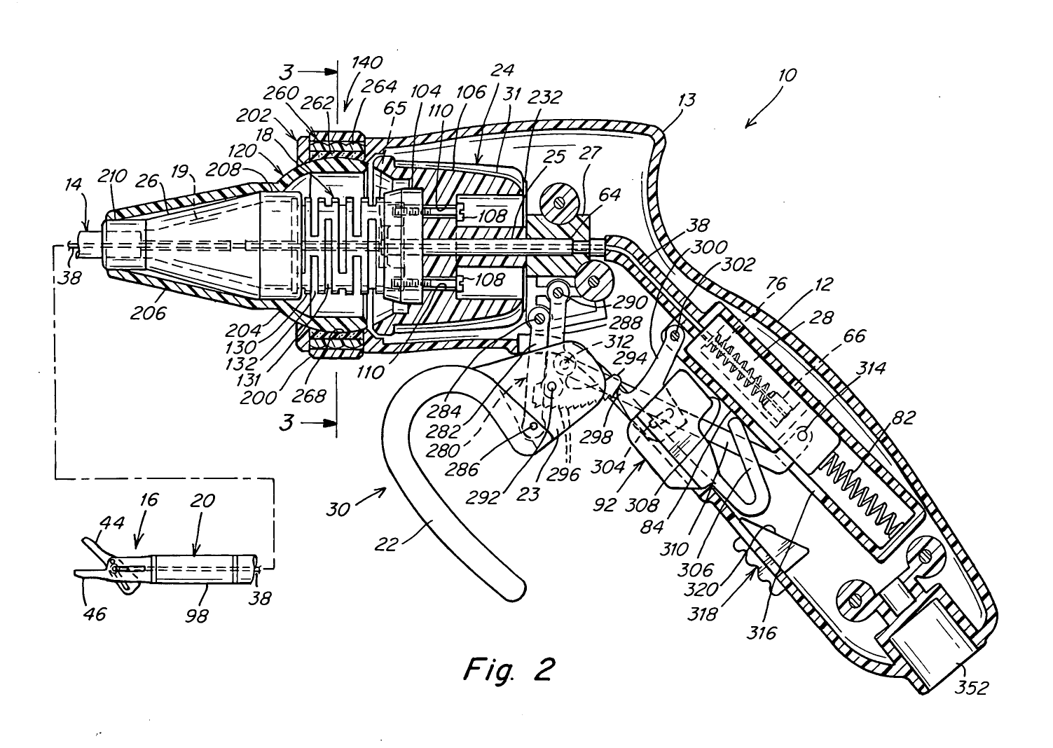

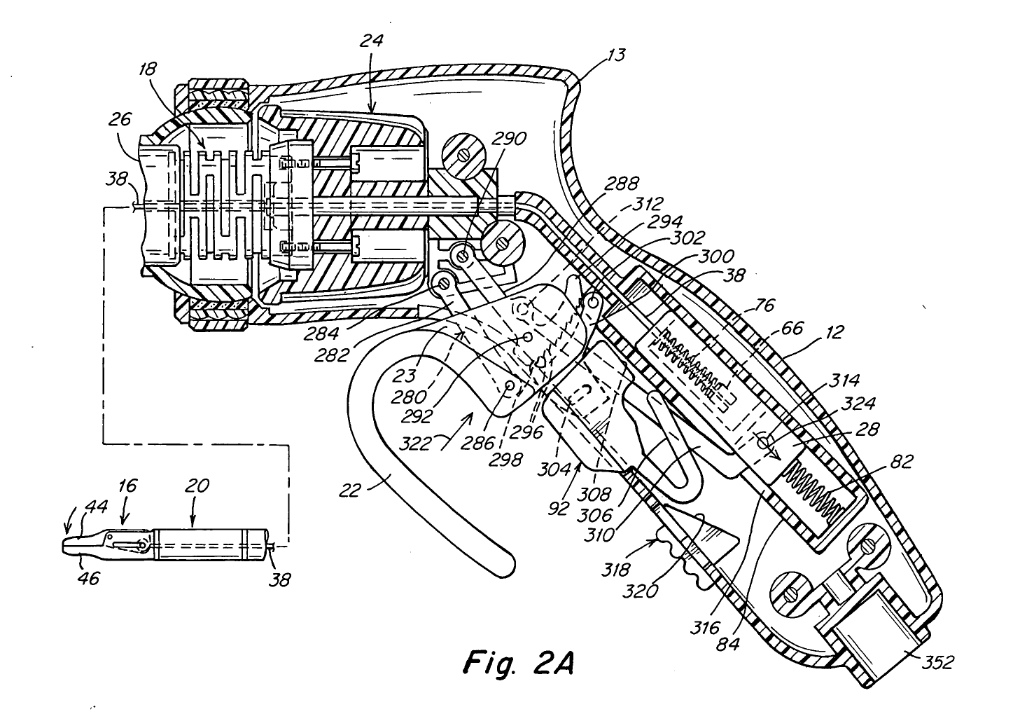

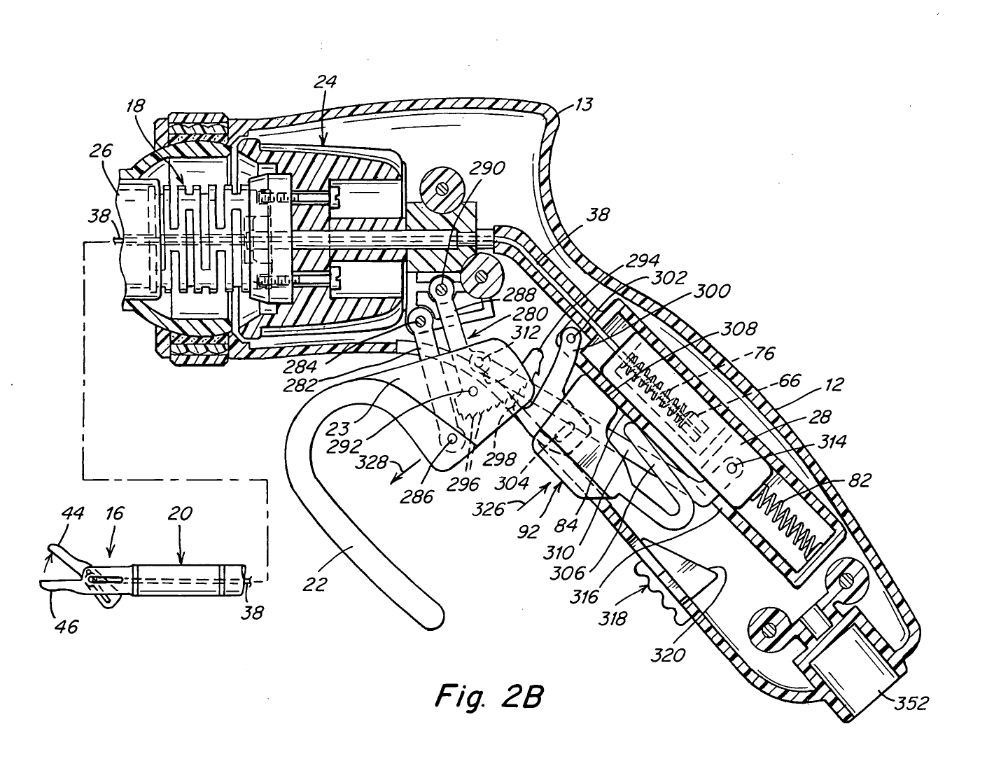

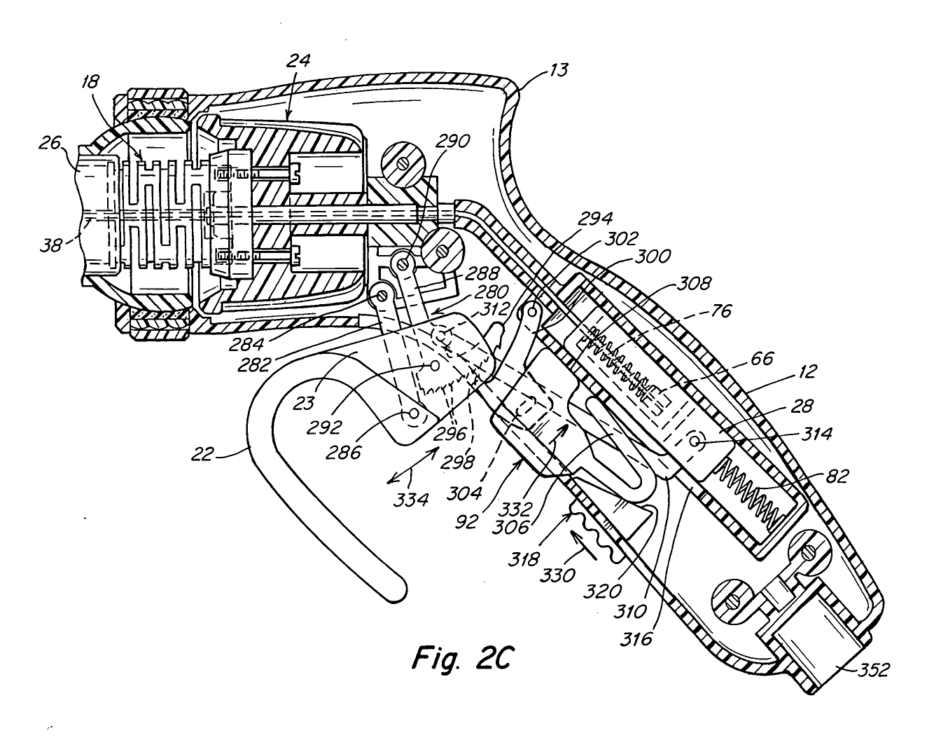

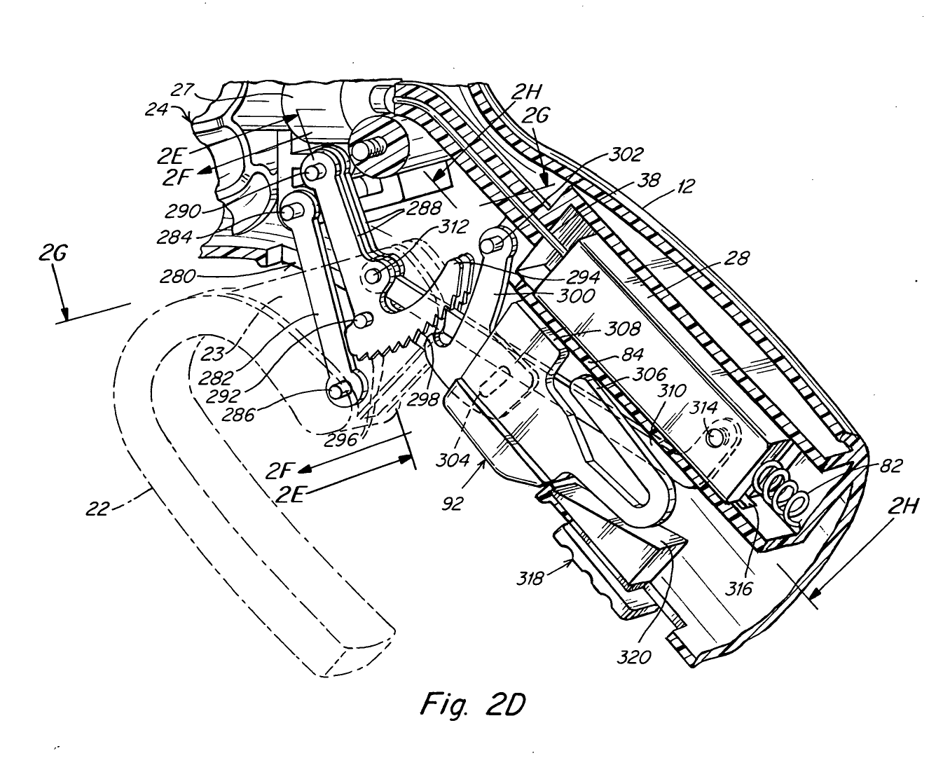

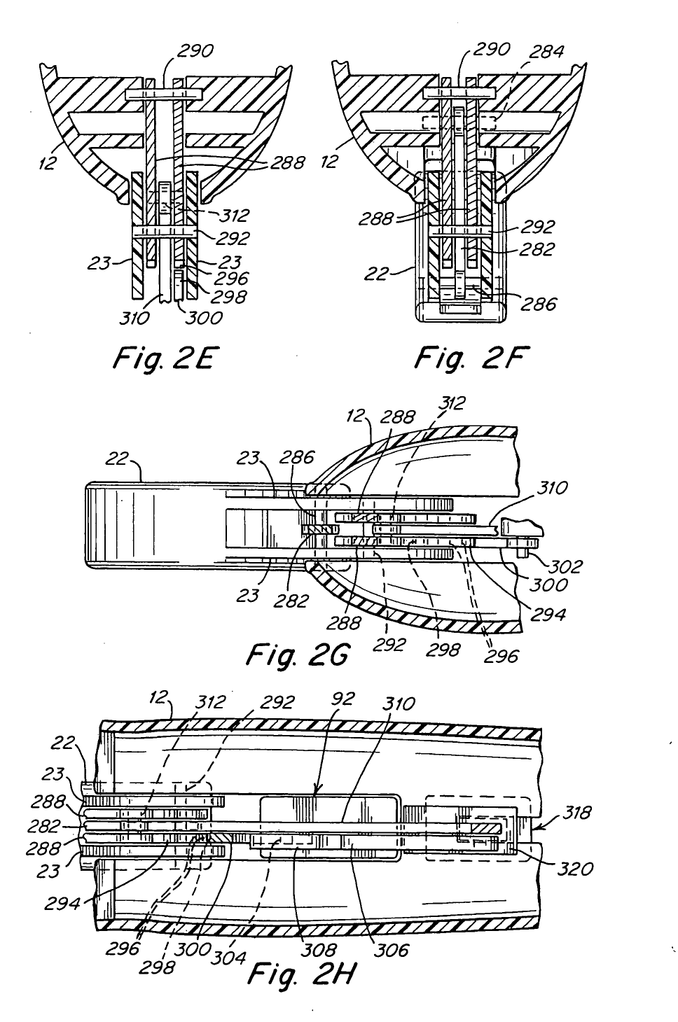

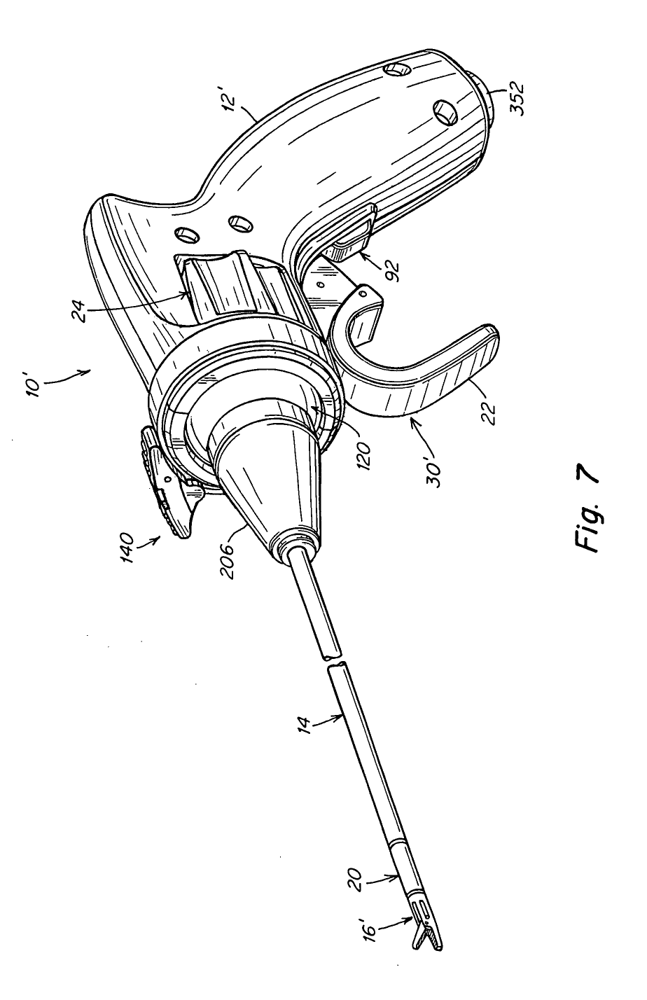

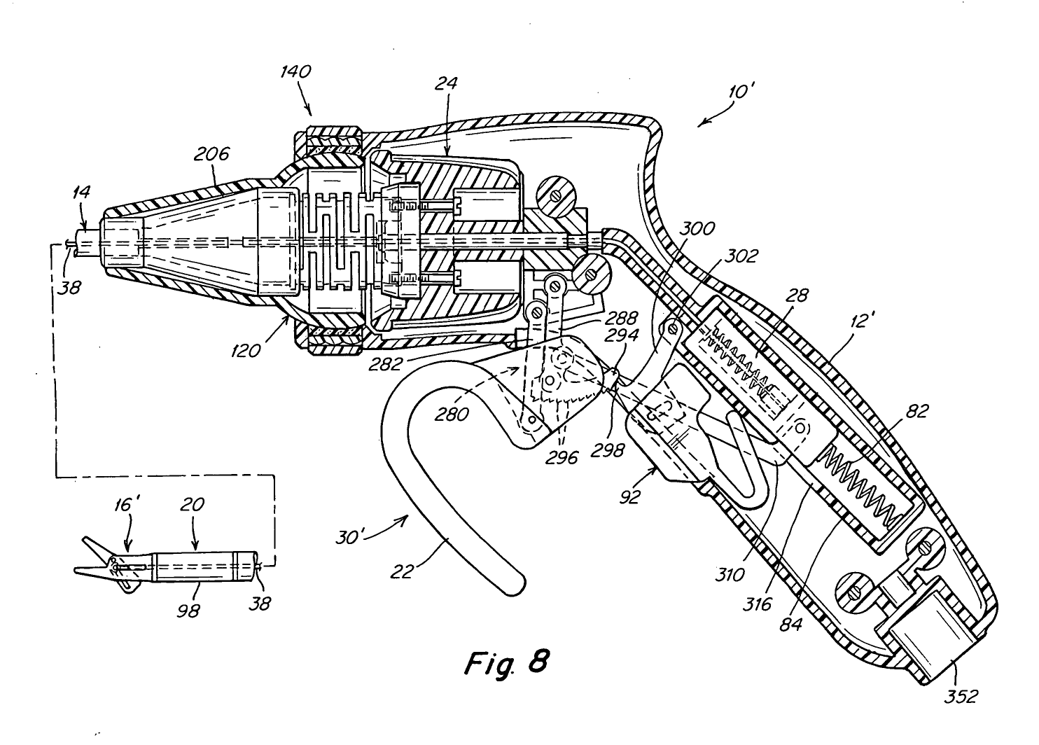

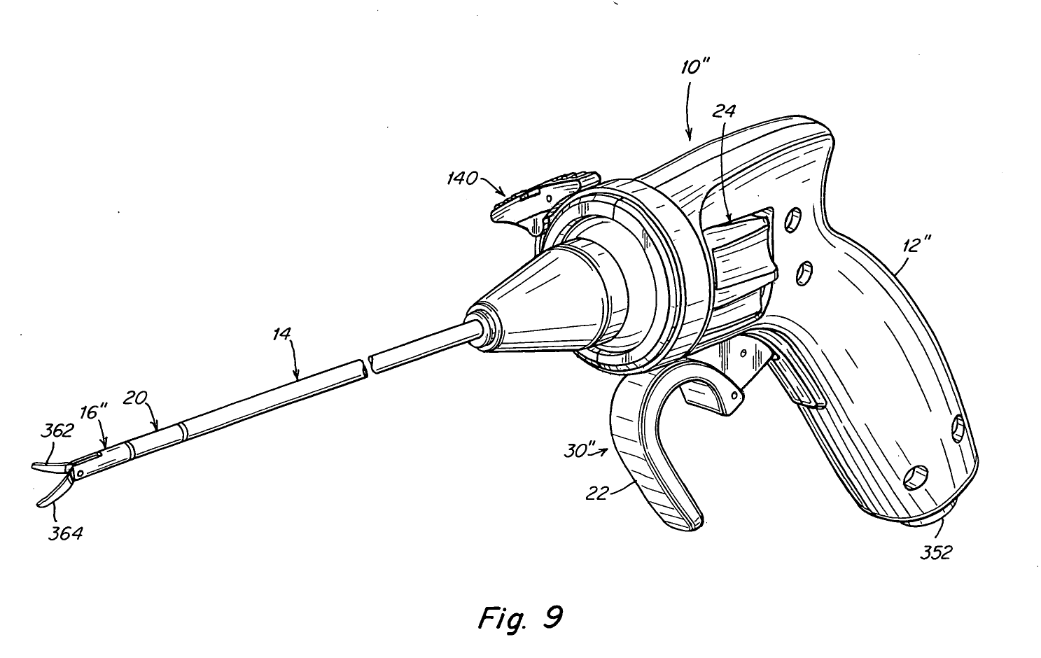

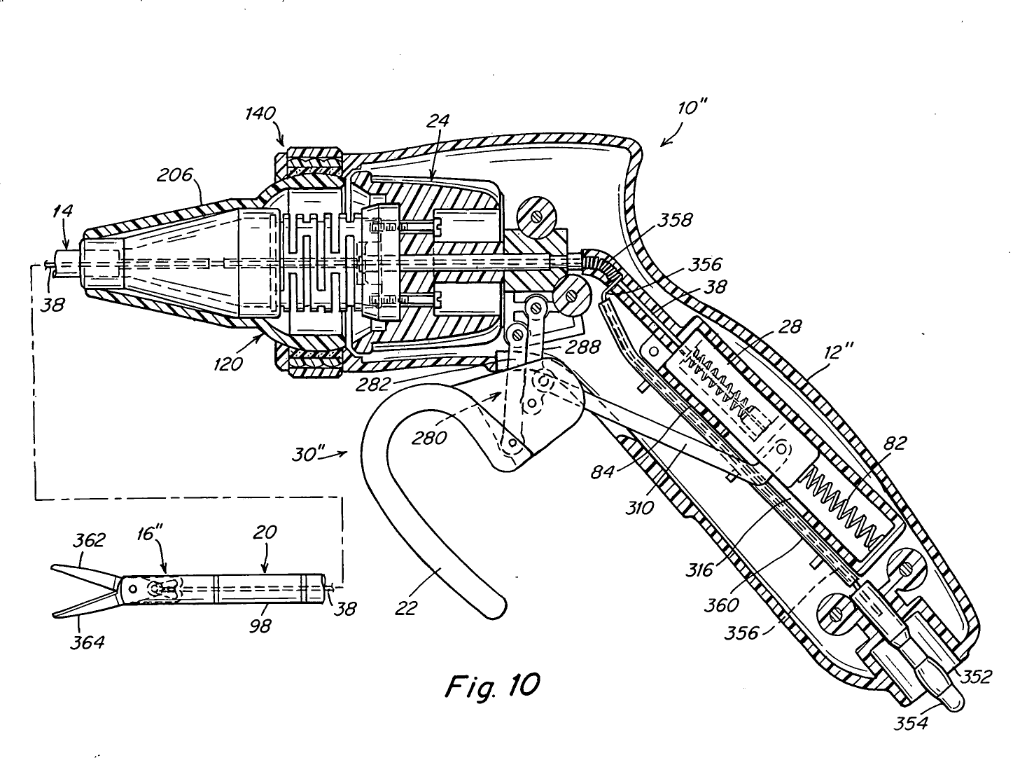

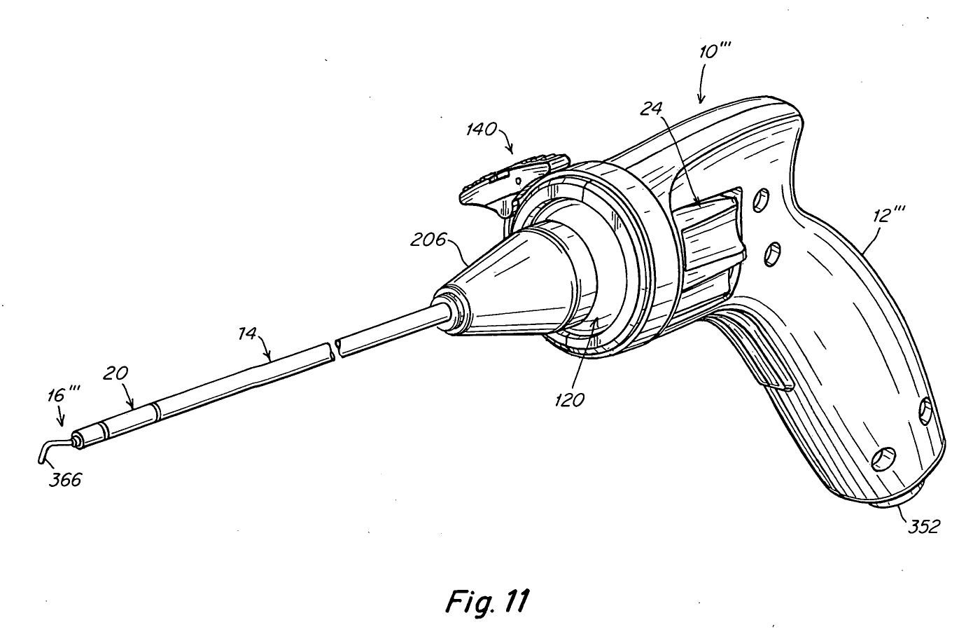

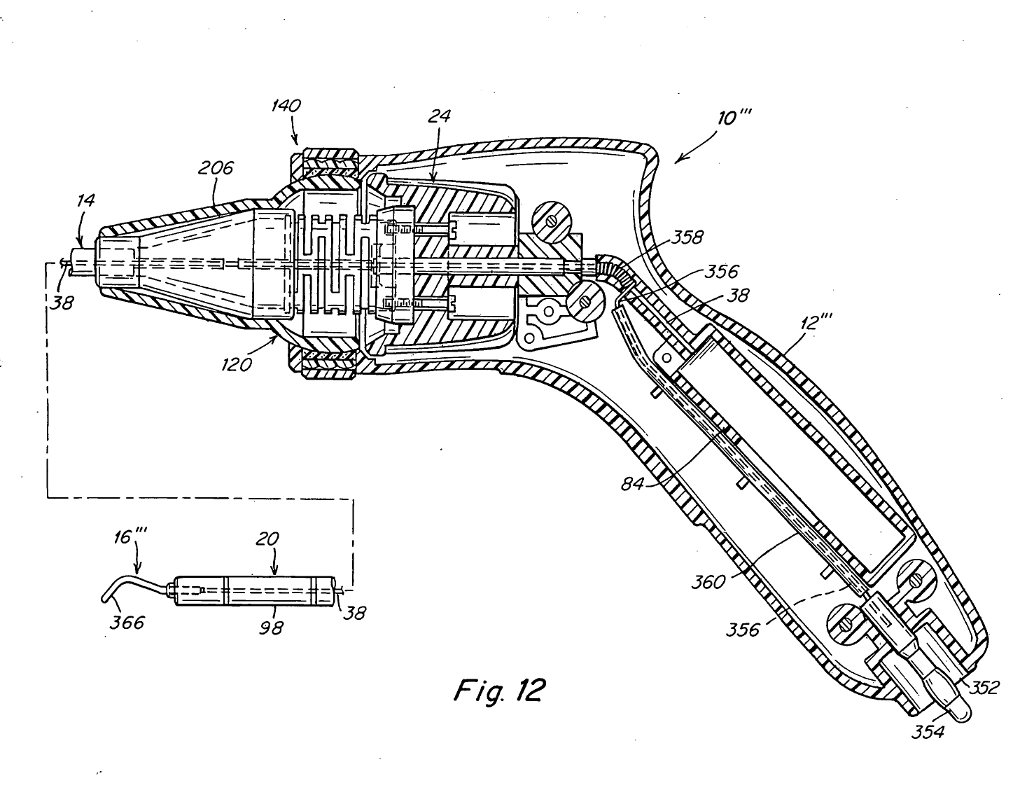

FIG. 1 is a perspective view of a preferred embodiment of the surgical instrument 10 of the present invention. FIGS. 2-6 provide further details of the embodiment of FIG. 1. Other versions of the instrument of the present invention are illustrated in FIGS. 7-12. FIG. 7 is a perspective view similar to that illustrated in FIG. 1 but of a second embodiment of the surgical instrument, illustrating a needle driver with only a ratcheting action, while FIG. 8 is cross-sectional side view similar to that illustrated FIG. 2 of this second embodiment of surgical instrument with the needle driver tool. FIG. 9 is a perspective view similar to that illustrated in FIG. 1 but of a third embodiment of the surgical instrument, illustrating a scissors without any ratcheting action, while FIG. 10 is a cross-sectional side view similar to that illustrated in FIG. 2 of this third embodiment of the surgical instrument with a scissors tool. FIG. 11 is a perspective view similar to that illustrated in FIG. 1 but of a fourth embodiment of the surgical instrument using a cautery tool, while FIG. 12 is a cross-sectional side view similar to that illustrated in FIG. 2 of this fourth embodiment of the surgical instrument with a cautery tool.

In the embodiment of FIG. 1 both the tool and handle motion members or bendable members are capable of bending in any direction. They are interconnected via cables (preferably four cables) in such a way that a bending action at the proximal member provides a related bending at the distal member. The proximal bending is controlled by a motion or deflection of the control handle by a user of the instrument. In other words the surgeon grasps the handle and once the instrument is in position any motion (deflection) at the handle immediately controls the proximal bendable member which, in turn, via cabling controls a corresponding bending or deflection at the distal bendable member. This action, in turn, controls the positioning of the distal tool.

The proximal member is preferably generally larger than the distal member so as to provide enhanced ergonomic control. In the illustrated embodiment the ratio of proximal to distal bendable member diameters may be on the order of three to one. In one version in accordance with the invention there may be provided a bending action in which the distal bendable member bends in the same direction as the proximal bendable member. In an alternate embodiment the bendable, turnable or flexible members may be arranged to bend in opposite directions by rotating the actuation cables through 180 degrees, or could be controlled to bend in virtually any other direction depending upon the relationship between the distal and proximal support points for the cables.

As has been noted, the amount of bending motion produced at the distal bending member is determined by the dimension of the proximal bendable member in comparison to that of the distal bendable member. In the embodiment described the proximal bendable member is generally larger than the distal bendable member, and as a result, the magnitude of the motion produced at the distal bendable member is greater than the magnitude of the motion at the proximal bendable member. The proximal bendable member can be bent in any direction (about 360 degrees) controlling the distal bendable member to bend in either the same or an opposite direction, but in the same plane at the same time. Also, as depicted in FIG. 1, the surgeon is able to bend and roll the instrument tool about its longitudinal axis to any orientation simply by rolling the axial rotation knob 24 about a rotation direction indicated in FIG. 1 by the rotation arrow R1. Moreover, further tool control is possible by a rotation of the entire instrument handle directly, as well as from the fulcrum effect controlled at the handle to pivot at the incision.

In this description reference is made to bendable members. These members may also be referred to as turnable members, bendable sections or flexible members. In the descriptions set out herein, terms such as endable section, endable segment, endable member, or urnable member refer to an element of the instrument that is controllably bendable in comparison to an element that is pivoted at a joint. The term movable member is considered as generic to bendable sections and joints. The bendable elements of the present invention enable the fabrication of an instrument that can bend in any direction without any singularity and that is further characterized by a ready capability to bend in any direction, all preferably with a single unitary or uni-body structure. A definition of a nitary or ni-body structure is—a structure that is constructed only of a single integral member and not one that is formed of multiple assembled or mated components—.

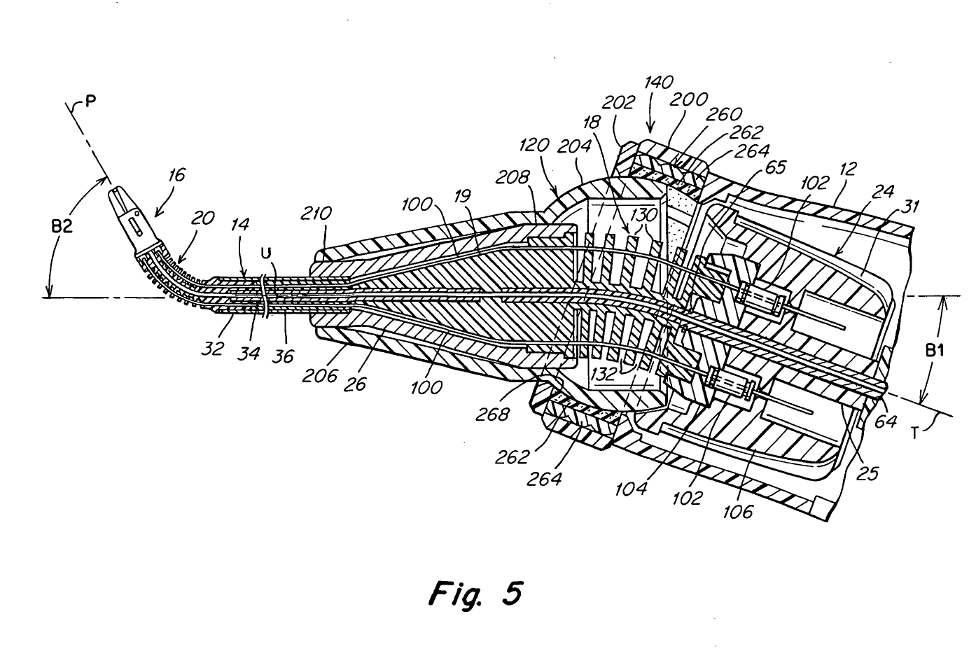

A definition of these bendable members is—an instrument element, formed either as a controlling means or a controlled means, and that is capable of being constrained by tension or compression forces to deviate from a straight line to a curved configuration without any sharp breaks or angularity—. Bendable members may be in the form of unitary structures, such as of the type shown herein in FIGS. 2 and 5 for the proximal and distal bendable members, may be constructed of engageable discs, or the like, may include bellows arrangements or may comprise a movable ring assembly. In FIG. 2 herein the unitary bendable proximal structure includes a series of alternating flexible discs 130 that define therebetween slots 132. A like structure can be used for the distal bendable member, per FIG. 5. A nitary or ni-body structure may be defined as one that is constructed for use in a single piece and does not require assembly of parts. Connecting ribs 131 are illustrated as extending between adjacent discs 130. Both of the bendable members preferably have a rib pattern in which the ribs are disposed at a preferred 60 degree variance from one rib to an adjacent rib. For several forms of bendable members refer to co-pending application Ser. No. 11/185,911 filed on Jul. 20, 2005; Ser. No. 11/505,003 filed on Aug. 16, 2006 and Ser. No. 11/523,103 filed on Sep. 19, 2006, all of which are hereby incorporated by reference herein in their entirety.

FIG. 1 shows one embodiment of the instrument of the present invention. Further details are illustrated in FIGS. 2 through 6. FIG. 1 depicts the surgical instrument 10 in a perspective view, as may occur during a surgical procedure. For example, the instrument may be used for laparoscopic surgery through an abdominal wall. For this purpose there is provided an insertion site (incision) at which there is disposed a cannula or trocar. The shaft 14 of the instrument 10 is adapted to pass through the cannula or trocar, so as to dispose the distal end of the instrument at the operative site. The end effector or tool 16 is depicted in FIG. 1. The embodiment of the instrument shown in FIG. 1 is typically used with a sheath 98 covering the distal member 20 to keep bodily fluids from entering the distal bendable member 20. Refer also to FIG. 5 which shows the end effector or tool 16 and the distal bendable member 20 without the surrounding sheath 98.

A rolling motion can be carried out with the instrument of the present invention. This can occur by virtue of the rotation of the rotation knob 24 relative to the handle 12 about a longitudinal shaft axis. This is represented in FIG. 1 by the rotation arrow R1. When the rotation knob 24 is rotated, in either direction, this causes a corresponding rotation of the instrument shaft 14. This is depicted in FIG. 1 by the rotational arrow R2. This same motion also causes a rotation of the distal bendable member and end effector 16 about an axis that corresponds to the instrument tip, depicted in FIG. 1 as about the longitudinal tip or tool axis P. In FIG. 1 refer to the rotational arrow R3 at the tip of the instrument. Refer also to the axis P in FIG. 5.

Any rotation of the rotation knob 24 while the instrument is locked (or unlocked) maintains the instrument tip at the same angular position, but rotates the orientation of the tip (tool). For a further explanation of the tip rotational feature refer to co-pending application Ser. No. 11/302,654, filed on Dec. 14, 2005, particularly FIGS. 25-28, which is hereby incorporated by reference in its entirety.

The handle 12, via proximal bendable member 18, may be tilted at an angle to the instrument shaft longitudinal center axis. This tilting, deflecting or bending is in three dimensions. By means of the cabling this action causes a corresponding bend at the distal bendable member 20 to a position wherein the tip is directed along an axis and at a corresponding angle to the instrument shaft longitudinal center axis. The bending at the proximal bendable member 18 is controlled by the surgeon from the handle 12 by manipulating the handle in essentially any direction including in and out of the plane of the paper in FIG. 1. This manipulation directly controls the bending at the proximal bendable member. For further descriptions relating to the bending and locking featues refer to co-pending application Ser. No. 11/528,134 filed on Sep. 27, 2006 and Ser. No. 11/649,352 filed on Jan. 2, 2007, both of which are hereby incorporated by reference in their entirety.

Thus, the control at the handle is used to bend the instrument at the proximal motion member to, in turn, control the positioning of the distal motion member and tool. The position of the tool is determined primarily by this bending or motion action and may be considered as the coordinate location at the distal end of the distal motion member. Actually, one may consider a coordinate axis at both the proximal and distal motion members as well as at the instrument tip. This positioning is in three dimensions. Of course, the instrument positioning is also controlled to a certain degree by the ability of the surgeon to pivot the instrument at the incision point or at the cannula or trocar. The orientation of the tool, on the other hand, relates to the rotational positioning of the tool, from the proximal rotation control member (knob 24), about the illustrated distal tip or tool axis P.

In the drawings a set of jaws is depicted, however, other tools or devices may be readily adapted for use with the instrument of the present invention. These include, but are not limited to, cameras, detectors, optics, scope, fluid delivery devices, syringes, etc. The tool may include a variety of articulated tools such as: jaws, scissors, graspers, needle holders, micro dissectors, staple appliers, tackers, suction irrigation tools and clip appliers. In addition, the tool may include a non-articulated tool such as: a cutting blade, probe, irrigator, catheter or suction orifice.

The surgical instrument of FIG. 1 shows one embodiment of a surgical instrument 10 according to the invention in use and may be inserted through a cannula at an insertion site through a patient skin. Many of the components shown herein, such as the instrument shaft 14, end effector 16, distal bending member 20, and proximal bending member 18 may be similar to and interact in the same manner as the instrument components described in the co-pending U.S. application Ser. No. 11/185,911 filed on Jul. 20, 2005 and hereby incorporated by reference herein in its entirety. Some other components shown herein, particularly at the handle end of the instrument may be similar to components described in the co-pending U.S. application Ser. No. 11/528,134 filed on Sep. 27, 2006 and hereby incorporated by reference herein in its entirety. Also incorporated by reference in their entirety are U.S. application Ser. No. 10/822,081 filed on Apr. 12, 2004; U.S. application Ser. No. 11/242,642 filed on Oct. 3, 2005 and U.S. application Ser. No. 11/302,654 filed on Dec. 14, 2005, all commonly owned by the present assignee.

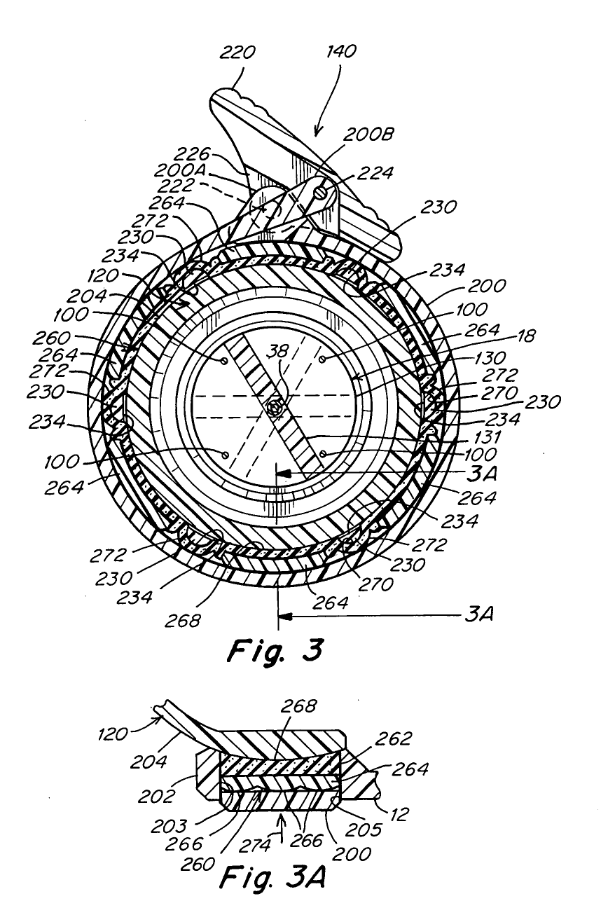

As illustrated in, for example, FIGS. 3 and 5, the control between the proximal bendable member 18 and distal bendable member 20 is provided by means of the bend control cables 100. In the illustrated embodiment four such control cables 100 may be provided in order to provide the desired all direction bending. However, in other embodiments of the present invention fewer or less numbers of bend control cables may be used. The bend control cables 100 extend through the instrument shaft 14 and through the proximal and distal bendable members. The bend control cables 100 may be constrained along substantially their entire length so as to facilitate both ushing and ulling action as discussed in further detail in the aforementioned co-pending application Ser. No. 11/649,352 filed on Jan. 2, 2007. The cables 100 are preferably constrained as they pass over the conical cable guide portion of the proximal bendable member, and through the proximal bendable member itself, as depicted in FIG. 5.

The locking means interacts with the ball and socket arrangement to lock and unlock the positioning of the cables which in turn control the angle of the proximal bending member and thus the angle of the distal bendable member and end effector. This lock control allows the surgeon one less degree of freedom to concentrate on when performing certain tasks. By locking the bendable sections at a particular position, this enables the surgeon to be more hands-free for controlling other degrees of freedom of the instrument such as manipulation of the rotation knob 24 and, in turn, orientation of the end effector.

The instrument shown in FIG. 1 is considered as of a pistol grip type. However, the principles of the present invention may also apply to other forms of handles such as a straight in-line handle. In FIG. 1 there is shown a jaw clamping or actuation means 30 that is comprised mainly of the lever 22. The actuation lever 22 controls the operation of the end effector 16.

In the instrument that is illustrated the handle end of the instrument may be tipped or deflected in any direction as the proximal bendable member is constructed and arranged to preferably enable full 360 degree bending. This movement of the handle relative to the instrument shaft bends the instrument at the proximal bendable member 18. This action, in turn, via the bend control cables 100, bends the distal bendable member in the same direction. As mentioned before, opposite direction bending can be used by rotating or twisting the control cables through 180 degrees from one end to the other end thereof.

In the main embodiment described herein, the handle 12 is in the form of a pistol grip and includes a horn 13 to facilitate a comfortable interface between the action of the surgeon hand and the instrument. The tool actuation lever 22 is shown in FIG. 1 pivotally attached at the base of the handle. The lever 22 actuates a linkage mechanism (see FIG. 2) that controls the tool actuation cable 38. The cable 38 controls the opening and closing of the jaws, and different positions of the lever control the force applied at the jaws.

The instrument 10 has a handle portion 12 and a shaft portion 14, as shown in FIG. 1. Many of the components of the instrument may be like that shown in Ser. No. 11/649,352 filed on Jan. 2, 2007, particularly as to the construction of the bendable members, instrument shaft, end effector, and rotation member. This includes means for enabling rotation of the shaft and proximal bendable member within bearings or bearing surfaces 208 and 210 (FIG. 2). The bearing surface 208 interfaces between a proximal end of the adaptor 26 and the ball 120, while the bearing surface 210 interfaces between the neck portion 206 and a distal end of the adaptor 26.

One of the characteristics of the embodiment shown herein is that the handle is constructed, along with the horn 13, to be more ergonomic and to accommodate different hand sizes particularly smaller hand sizes. This includes, inter alia, a shortened horn, an improved construction and shape of the tool actuation means 30 and the improved shape and contour of the base of the handle 12. The rotation knob 24 is also made longer as illustrated in FIG. 1 thus providing better access and control to the rotation knob by the user.

With reference to FIGS. 1 and 2 there is illustrated a medical instrument 10 that is basically comprised of a handle 12, proximal bendable member 18, instrument shaft 14, distal bendable member 20 and tool or end effector 16. The shaft 14 may be considered as having a longitudinal axis U. Similarly, the handle 12 may be considered as having a longitudinal axis T, and the end effector 16 may be considered as having a distal tip axis P. In FIG. 1 all of the axes U, T and P are in-line, while FIG. 5 illustrates the position of these same axes when the instrument is in a bent state. A bending of the handle 12 with respect to the shaft 14 is shown in FIG. 1 by virtue of the double arrows that show an angle B1. This bending at the proximal bendable member causes a corresponding bending at the distal bendable member resulting in a bend indicated by the double arrows at an angle B2 between the instrument shaft 14 and end effector 16. As illustrated in FIG. 5, when the handle is bent downwardly, the end effector bends upwardly. The ratio of bending angle is determined by the ratio of the diameters of the proximal bending member 18 and the distal bending member 20, which, in turn, determines the distance each cable 100 is push/pulled as best illustrated in FIG. 5.

With further reference to FIG. 1, there are illustrated several different instrument motions indicated by rotational arrows. Rotation arrow R1 represents the rotation of the rotation knob 24 about handle axis T. This action, in turn, causes a rotation illustrated by rotation arrow R2 of the shaft about axis U. The rotation knob 24 supports the proximal bendable member 18 which, in turn, supports the instrument shaft 14. The rotation R2 is transmitted to rotation R3 of the end effector 16 about axis P by way of the distal bendable member 20. Cables 100 are anchored at one end at end effector 16 and at the opposite end at crimps 102 (see FIG. 5) at the rotation knob 24. The cables control, in a push and pull manner, the end effector 16 as it is rotated to keep axis P at its preset angle that is fixed by the angle locking means 140. The distal bendable member 20 is generally smaller in diameter than the proximal bendable member 18 and has discs, ribs and slots similar to the discs 130, ribs 131 and slots 132 seen in FIG. 2 for the proximal bendable member. The distal bendable member 20 also preferably has a sheath 98 encasing it to prevent bodily fluids from infiltration.

When the instrument is in use bending forces are applied to the handle 12 and accordingly to the shaft 14 as the instrument is manipulated through a portal in the patient. Many times the forces that are imposed during a surgical procedure can be substantial, possibly resulting in a creeping or slippage, in the locked state, between the ball 120 and the hub 202. The possible slippage is represented in FIG. 1 by respective arrows M and R4. The double arrows M represent an oscillation movement and the single arrow R4 represents a rotation movement. Refer also to aforementioned Ser. No. 11/649,352 (Publication No. 2008/0065116) and the hub 202 that is clamped directly by the cinch ring 200.

There are several improvements embodied in the disclosed instrument constructed in accordance with the principles of the present invention. One objective of the present invention is to provide a stronger angle locking device so as to avoid or at the least minimize any slippage of the angle locking means 140. To accomplish this objective the invention uses a resilient compression ring 260 that is keyed to the hub 202 and disposed between the cinch ring 200 and the ball 120 as will be discussed later. Another feature of the present invention relates to an improved paddle-shaped release/lock lever 220 is also embodied in the instrument and mounted with the cinch ring 200. Still another feature of the present invention relates to a new ergonomic jaw clamping member (lever) 22 that has also been incorporated into the instrument as best illustrated in FIG. 2.

In connection with the jaw clamping mechanism, the lever 22 is attached to the handle 12 by a multi-bar linkage 280 that includes a single forward link 282 connected at opposite end pivots 284, 286; and a pair of rearward links 288 commonly connected at opposite end pivots 290, 292. The links 288 are spaced apart substantially in parallel to each other and separated by approximately the thickness of the link 310, as is depicted in FIG. 2D. At least one of the links 288 has a ratchet arm 294 that engages pawl 298 that is formed on the lower end of link 300. FIG. 2D shows a ratchet arm on both links 288, but only one ratchet arm engages with the pawl 298 (simpler in manufacturing to not have to provide two different configuration links 288).

The links 288 are coupled by means of pivot pin 312 to one end of elongated link 310 which is attached at its opposite end to the slider 28 at pivot pin 314. The slider 28 is biased by spring 82 to a distal t rest position where the end effector jaws may be forced to an open position. The spring 82 may be optional. When the lever is fully squeezed to apply full pressure and lock-in the jaws, the link 310 pushes the slider proximally thus locking the jaws on a needle or other implement. This leaves the surgeon fingers free to work the rotation knob 24. Squeezing the release button 92 against pressure from spring arm 306 pivots button 92 counterclockwise releasing lever 22 and the end effector jaws.

As mentioned previously, the jaw actuation means 30 is primarily comprised of an actuation lever 22 which is attached to the handle 12 by the linkage 280. The forward links 282 is pivotally attached respectively to the handle at pivot pin 284 and to the lever 22 at pivot pin 286. The pair of links 288 are respectively coupled to the handle 12 at pivot pin 290 and to the lever 22 at pivot pin 292. At least one of the links 288 has a ratchet arm 294 that is formed with a ratchet surface that includes numerous teeth 296. FIG. 2D depicts the teeth 296 engageable with the pawl 298 that is formed on the lower end of link 300 which is pivotally attached to the handle 12 at pivot pin 302. The link 300 is insert-molded to the release button 92 at 304.

The tool actuation mechanism at the handle also includes a spring arm 306 which is formed as a living hinge on the top of the release button 92. This spring arm 306 bears against the underside of the slideway 84 which houses the slider 28. The spring arm 306 normally biases the pawl 298 against the teeth 296 of the ratchet arm 294. A stop 308 is formed on the upper surface of the release button 92 to limit the inward travel of the release button. The stop 308 engages the underside of the slideway 84 when the button 92 is fully depressed as is illustrated in FIGS. 2B and 2C.

Also mounted adjacent to the release button 92 is a ratchet disengage slide 318 with a wedge 320 that is mounted with the slide 318. The slide 318 can be used to move the release button 92 to the position shown in FIG. 2C when it might be desirable to disengage the ratchet and pawl and allow the lever 22 to be free floating during use. The links 288 are coupled by pivot pin 312 to one end of link 310 which is attached to slider 28 at pivot pin 314 after passing through slot 316 in the slideway 84.

FIG. 2 illustrates the instrument in an un-actuated state with the lever 22 in a position away from the body of the handle. FIG. 2A illustrates the actuation lever 22 being fully depressed in the direction of arrow 322 to close the jaws of the end effector. As the links 288 swing inward to the handle, link 310 is pushed in the direction of arrow 324 and the slide 28 moves proximally against spring 82, pulling cable 38 and closing the jaws against a tool (not shown in FIG. 2A). A barrel or crimp 66 and spring 76 arrangement in slot 78 of slider 28 accommodates different thicknesses of tool and rotation of the end effector. In this regard refer to a like actuation mechanism shown in either Ser. No. 11/528,134 or Ser. No. 11/649,352, both of which have been incorporated by reference herein. The springs 76 and 82 shown in the drawings may be replaced by other biasing means such as a resilient compressible tube member. The spring 76 is considered as a force limiting spring.

The spring pressure is provided by spring 76 acting against crimp 66 as described to accommodate different thicknesses of objects grasped. The lever 22 can be held in its most squeezed position by the ratchet and pawl. This effectively locks the jaws on an object, freeing the surgeon fingers to work other features of the instrument.

Although the lever 22 is shown fully depressed, it may be retained in any position between those shown in FIGS. 2 and 2A. The ratchet and pawl retain the lever in the furthest position to which it is squeezed, until the release button 92 is depressed in the direction of the arrow 326 as shown in FIG. 2B. When the release button 92 is depressed, against the bias of the spring arm 306, the release button 92 moves until the stop 308 contacts the bottom surface of the slideway 84. This action pulls the pawl 298 out of engagement with teeth 296 of the ratchet arm 294. The depressing of the release button 92 actually causes a slight counter clockwise movement of the release button to thus separate the pawl 298 from the ratchet teeth 296. At the same time the spring 82 pushes the slider 28 back toward its rest position, as well as the actuation lever 22 in the direction of arrow 328 via link 310 and multi-bar linkage 280.

In some circumstances it is desirable to be able to freely work the jaws of the end effector without a ratcheting action. To accomplish this, the ratchet disengage slide 318 may be pushed in the direction of arrow 330, as depicted in FIG. 2C. When slide 318 is moved toward the release button 92 the ramp 320 pushes up on the spring arm 306 and pivots the release button counter clockwise in the direction of arrow 332 about the pivot pin 302. This action has the effect of forcing wedge 320 against the release button 92 moving it in the direction of arrow 332 until stop 308 contacts the underside of the slideway 84. This means that pawl 298 also pivots counter clockwise about the pivot pin 302 since the release button and the link 300 are conjoined at the co-extrusion 304. In this position, the pawl 298 is disengaged from the ratchet teeth 296 and the actuation lever 22 is free to move in the direction of double headed arrow 334 without any ratcheting action. A detent (not shown) may be used on the slide 318 in the released position until it is manually released.

FIG. 2 shows the jaws 44, 46 in what may be considered their at rest position with the lever un-actuated. FIG. 2A shows the lever 22 being fully squeezed in the direction of arrow 322 resulting in a movement of the slider 28 in the direction of arrow 324, closing jaws 44, 46 and pawl 298 engaging and holding ratchet teeth 296.

FIG. 2B shows the release button 92 being squeezed in the direction of arrow 326 which releases the pawl 298 from teeth 296 allowing the release of lever 22 in the direction of arrow 328 and the jaws to open by the bias of spring 82. FIG. 2C shows the ratchet disengage slide 318 slid in the direction of arrow 330 which pushes in release button 92 in the direction of arrow 332 which holds pawl 298 out of engagement with teeth 298 and allows the lever 22 to free-float (double headed arrow 334) and the jaws to open and close against finger-grip pressure. FIG. 2D is a fragmentary perspective view of the jaw actuation means 30 in the position seen in FIG. 2C and best shows the relationship of the multiple bar linkage 280. Although there are two ratchet arms 294, preferably only one is used to engage pawl 298 and link 300.

As illustrated in FIGS. 1 and 2 the finger grip portion of the rotation knob 24 with indents 31 has been lengthened for easier manipulation. The rotation knob 24 is rotationally mounted on center wire conduit 64 which is mounted on the boss 298 by the hub 25 and longitudinally secured to boss 298 by E-ring 65 to maintain a gap 232 to prevent interference between the rotational knob 24 and handle housing. The proximal bending member 18 is seated in the rotational knob at the distal end of the rotation knob, and the distal conical end 19 is seated in adapter 26. The length of the proximal bendable member may be shorter than in an instrument such as shown in Ser. No. 11/649,352. There are thus fewer discs 130 therefore somewhat simplifying the instrument, and allowing the ball member 204 to be smaller. The conical portion 19 is seated in adapter 26 to which the proximal end of shaft 14 is mounted. As shown in FIG. 5, the shaft 14 has an outer shaft tube 32, inner shaft tube 34 and shaft filler 36 with lumens or passages for cables 38 and 100. The distal end of the shaft 14 supports the distal bending member 20 to which the end effector 16 is attached. The adapter 26 is free to rotate within the neck 206 of ball 120 at bearing surfaces 208, 210.

In addition to making the rotation knob longer, another improvement in accordance with the present invention relates to providing the rotation knob in two separate portions. This simplifies assembly of the instrument. The rotational knob is made up of an inner knob 104 and an outer knob 106 that are held together by screws 108. As best illustrated in FIGS. 2 and 5, the proximal bending member 18 is seated in the inner knob 104 and the cables 100 pass through holes in the inner knob and are crimped at 102. The inner knob 104 is seated or nested with the outer knob 106 and screws 108 pass through clearance holes 110 in the outer knob and into threaded holes in the inner knob to clamp the two knob portions together and clamp the crimps 102 in between. Refer also to FIG. 5 which illustrates a cross-sectional view through, not only the rotation knob, but also the proximal bendable member 18 and the adaptor 26. The proximal end of the proximal bendable member 18 is nested within an annular groove in the inner knob portion 104.

There are two fewer discs 130 in the proximal bending member than in the past instrument. The conical portion 19 is seated in adapter 26 to which the proximal end of shaft portion 14 is mounted. The shaft portion 14 has an outer shaft tube 32, inner shaft tube 34 and shaft filler 36 with lumens or passages for cables 38 and 100 as can be seen in FIG. 5. The distal end of the shaft portion 14 supports the distal bending member 20 to which the end effector 16 is attached. The end effector in this first embodiment is depicted as a grasper but other configurations of end effector may also be used. The adapter 26 is free to rotate within the neck 206 of ball 120 at bearing surfaces 208, 210.

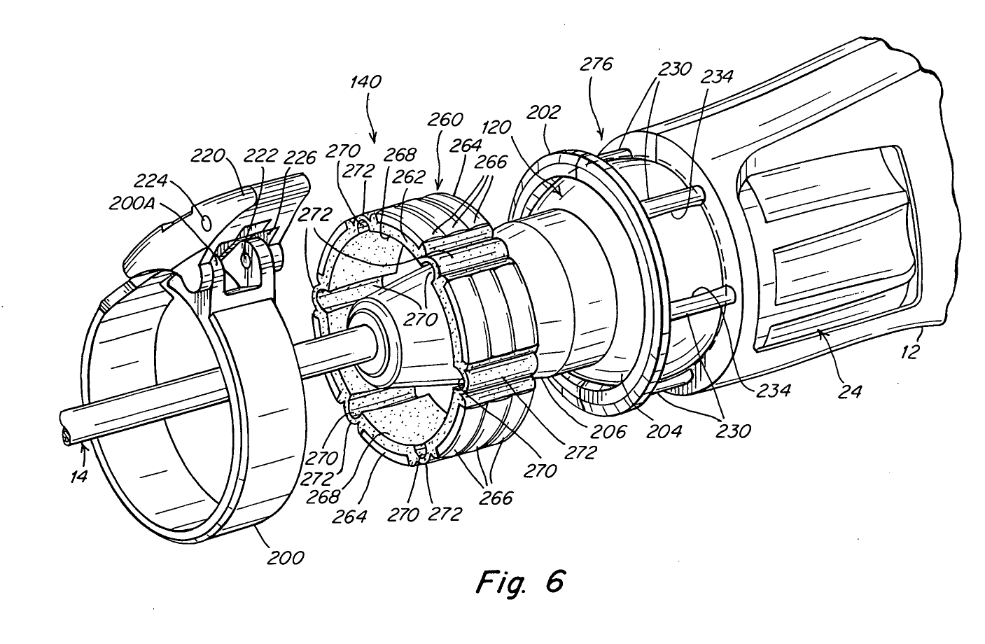

The improved angle locking means 140 is now described. As can be best seen in FIG. 6, instead of the cinch ring clamping split hub segments to the ball 120 as in, for example, the instrument shown in Ser. No. 11/649,352, the hub 202 is connected to the handle 12 by struts 230 with spherically shaped inner facing surfaces 234 which along with the inner facing surface of the hub 202 form a retaining socket for the ball 120. A resilient compression ring 260 is seated in the gap 276 between the hub 202 and the handle body. The ring 260 is keyed to the struts 230 by means of channels 270 engaging with respective struts 230 to prevent any rotational (circumferential) movement between the ring 260 and the handle 12. The resilient compression ring 260 may be considered as basically including the relatively soft resilient closed annular member 262 and a plurality of solid stiff segments 264 that are spacedly disposed about the outer surface of the relatively soft resilient closed annular member 262.

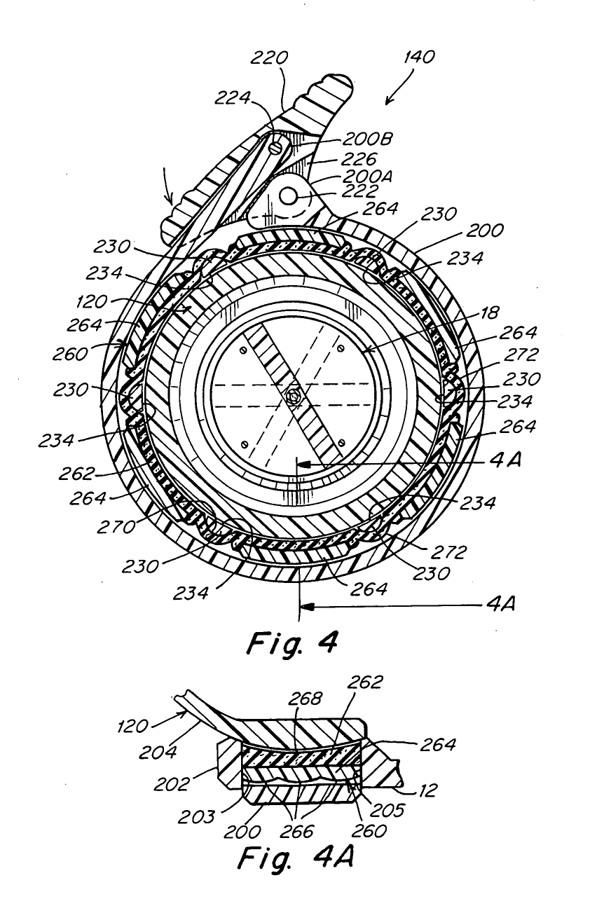

The soft resilient member 262 may be considered as including separate ring shape segments that are connected by a like number of hinge sections 272. The resilient soft rubber portion 262 of the compression ring 260 is formed in a ring shape of a plurality of segments (six such segments disclosed in FIG. 6, but more or less than that amount can be used) connected by hinge sections 272 that define respective channels 270 that are interfaced or interlocked with the struts 230. The inner surface of each of the segments is spherical to match the surface 204 of the ball 120, as is shown in FIGS. 5 and 6. The resilient hinge sections 272 have a memory that makes them act as springs against the spacers or struts 230 to lift the segments away from the surface of the ball 120 when the cinch ring 200 is released, as shown in FIGS. 4 and 4A. FIGS. 3 and 3A, on the other hand, shows the locking mechanism in a locked position.

On the outside surface of the compression ring 260 are attached six stiff plastic segments 264 each with circumferential ribs 266. Each of these segments 264 overly the aforementioned compression ring segments. The stiff segments 264 apply an even pressure as indicated by the arrow 274 in FIG. 3A across the outer surface of the six resilient segments of the soft resilient member 262. The stiff plastic segments 264 and their associated ribs 266 provide minimal resistance as the cinch ring slides along them as the cinch ring 200 is tightened. When the cinch ring 200 is tightened the resilient compression ring 260 is compressed against the ball 120 and provides an excellent frictional lock against any slippage or creep between the resilient compression ring 260 and the ball 120. The compression ring 260 is preferably relatively loosely secured to the handle 12 by hub 202 and in particular about the struts 230. The channels 270 essentially interlock with the struts 230. In the position illustrated in FIGS. 3 and 3A the angle locking is in the locked position wherein the cinch ring 200 is tightened about the compression ring 260 providing a locking in at the angle B1.

When released, the cinch ring 200 is retained in the gap 276 by shoulder 203 on the hub 202 and the shoulder 205 on the distal end of the handle as can be seen in FIG. 4A. The cinch ring has two ends 200A and 200B. Similar to the instrument shown in Ser. No. 11/649,352, there is a release/lock lever 220 in the shape of a paddle attached to end 200A by pin 222. There is a slot 226 on the inside of the lever to receive the end 200A. The other end 200B of the cinch ring is attached to the lever 220 by pin 224. When the lever is flipped to the position illustrated in FIG. 3, the lever pulls the end 200B about the axis of pin 222 until pin 224 is in an over center arrangement as seen in FIG. 3 and the tension of the cinch ring keeps the lever firmly in the locked position. When the lever is flipped to the position illustrated in FIG. 4, the cinch ring is relaxed enough to allow the resilient hinge sections 272 to lift the six segments of the compression ring 260 away from the surface of the ball allowing the ball to be freely rotated in its socket.

An important improvement of the instrument of the present invention relates to the use of a member that is at least partially resilient as described herein in connection with the ball and socket arrangement along with the locking cinch ring. The resilient ring arrangement actually allows a higher degree of friction between the resilient ring and the ball member. Moreover, this is accomplished with a minimum of cinch ring force applied. This also makes the use of the instrument more user friendly. The ball member is somewhat smaller than earlier versions decreasing the inter-surface contact area, and thus decreasing the amount of force required in order to lock the instrument in place. This also makes the ball and cinch arrangement less sensitive to dimensional tolerances.

Reference is now made to additional embodiments of the instrument shown in FIGS. 7-12. FIG. 7 is a perspective view similar to that illustrated in FIG. 1 but of a second embodiment of the surgical instrument, illustrating a needle driver with only a ratcheting action, and related FIG. 8 is a cross-sectional side view of the embodiment shown in FIG. 7 with a needle driver tool. FIG. 9 is a perspective view similar to that illustrated in FIG. 1 but of a third embodiment of the surgical instrument, illustrating a scissors without any ratcheting action, and related FIG. 10 is a cross-sectional side view of the FIG. 9 embodiment of the surgical instrument with a scissors tool. FIG. 11 is a perspective view similar to that illustrated in FIG. 1 but of a fourth embodiment of the surgical instrument using a cautery tool, and related FIG. 12 is a cross-sectional side view of the FIG. 11 embodiment of the surgical instrument with a cautery tool.

In the three additional embodiments shown in FIGS. 7-12 herein like reference numbers are used where appropriate to describe like instrument components. For the most part in these additional embodiments a major part of the instrument stays the same, but some of the features illustrated in the first embodiment of FIG. 2 have been removed depending upon the particular end effector that is to be used with the instrument. Because of the use of many like components in these additional embodiments, additional detail may be left off with reliance being made from the first embodiment described herein.

FIGS. 7 and 8 illustrate a second embodiment 10 of surgical instrument in which the end effector 16 is shown to be a needle driver. When used as a needle driver, it is desirable to have a ratcheting action only in the jaw clamping means 30 Thus the ratchet disengage slide 318 shown and described previously in connection with the FIG. 2 embodiment, is removed from the handle and the slot in the handle 12 through which it protruded is now closed with plastic as shown in the housing of the handle 12 of FIG. 8. In this embodiment, even though only a ratcheting action is used, it is understood that the instrument does have the release feature as controlled from the release button 92 which was previously described. Refer to FIGS. 7 and 8 for the release button 92.

FIGS. 9 and 10 illustrate a third embodiment 10 of surgical instrument in which the end effector 16 is shown as a scissors. When used as a scissors, it is desirable to have a free floating jaw clamping means 30. Thus the ratcheting mechanism and the release button have been removed in this embodiment and the corresponding slot in the handle for the release button has been closed with plastic as shown in the handle 12 of FIG. 10. In this embodiment the actuation linkage has been simplified as no ratchet action is now used.

In this third embodiment the rear links 288 have been modified by deleting the ratchet arms 294 and the link 300 and release button 92 have been removed as well. Still another version of the instrument might include a dissector tool (not shown) as an end effector. Either the scissors or dissector may be additionally used as a cautery tool by the addition of a banana plug connector 354 installed in the socket 352 at the base of the handle. Refer to FIG. 10 that illustrates the location of the banana plug connector 354 and associated socket 352. The connector may be plugged into a jack connected to an electrical generation source that can heat the end effector by induction to a temperature suitable for cauterization of tissue. The banana plug connector 354 is electrically connected to the end effector by means of cable 38 and connecting wire 356 which is wrapped around cable 38 at 358 and is protected by insulating sheath 360 as best seen in FIG. 10. The sheath 360 may extend from the banana plug connector to the area of the coil 358, and another section thereof may extend distally from the coil 358 to the end effector.

Reference is now made to FIGS. 11 and 12 that illustrate a fourth embodiment 10 of surgical instrument in which the end effector 16 is shown as a bent wire. When used with just a bent wire, there is no need for a jaw actuation means and thus the corresponding slot for the same has been filled in with plastic as shown in handle 12 of FIG. 12. The jaw actuation means 30, slider 28 and related hardware have all been removed and the cable 38 terminated at the entrance of the slideway 84.

Having now illustrated a certain number of embodiments of the present invention, it should be apparent to those skilled in the art that numerous other embodiments and modifications thereof are contemplated as falling within the scope of the present invention as defined by the appended claims. For example, although a certain number of embodiments have been illustrated, particularly for using different types of tools or end effectors, it is contemplated that many other embodiments can be envisaged for providing any one of a number of different end effectors.

What is claimed is:

1. A surgical instrument comprising:
an instrument shaft having proximal and distal ends;
a tool disposed from the distal end of the instrument shaft;
a control handle coupled from the proximal end of the instrument shaft;
a distal bendable member for coupling the distal end of said instrument shaft to said tool;
a proximal bendable member for coupling the proximal end of said instrument shaft to said control handle;
actuation means extending between said distal and proximal bendable members for coupling motion of said proximal bendable member to said distal bendable member for controlling the positioning of said tool;
and a locking mechanism for fixing the position of the tool at a selected position;
said locking mechanism including a ball and socket arrangement disposed about said proximal bendable member and a locking member for locking said ball and socket arrangement and having locked and unlocked states;
said ball and socket arrangement including a compression ring supported from the control handle, having an outer surface for support of the locking member thereabout and having an inner surface defining an at least partially spherical shaped socket;
said ball and socket arrangement further including a hollow ball member having an internal hollow chamber and an outer at least partially spherical shaped surface that mates with the at least partially spherical shaped socket.

2. The surgical instrument of claim 1 wherein the hollow ball member is supported within said socket and constructed and arranged with at least a portion of the proximal bendable member disposed in the internal hollow chamber of the hollow ball member.

3. The surgical instrument of claim 2 wherein the compression ring includes an annular resilient base member, and a plurality of stiffener segments that are disposed about the annular resilient base member.

4. The surgical instrument of claim 3 wherein the annular resilient base member includes a plurality of spacedly disposed hinges that define therebetween a plurality of support segments.

5. The surgical instrument of claim 4 wherein the plurality of stiffener segments are disposed over the respective plurality of support segments.

6. The surgical instrument of claim 5 wherein each stiffener segment has, on a top surface thereof, a series of ribs.

7. The surgical instrument of claim 6 wherein the ribs are spaced apart and extend circumferentially.

8. The surgical instrument of claim 4 including at least a hub for supporting the compression ring.

9. The surgical instrument of claim 8 including a plurality of struts for supporting the hub from the control handle.

10. The surgical instrument of claim 9 wherein each hinge includes a passage for receiving a respective strut.

11. The surgical instrument of claim 1 further including a rotation means disposed adjacent the control handle and rotatable relative to the control handle for causing a corresponding rotation of the instrument shaft and tool.

12. The surgical instrument of claim 11 wherein said rotation means comprises a rotation knob that is adapted to rotate the tool about a distal tool roll axis and said rotation knob is disposed between said control handle and proximal bendable member.

13. The surgical instrument of claim 1 wherein said control handle comprises a pistol grip handle having an engagement horn to assist in holding the handle.

14. The surgical instrument of claim 13 including an actuation lever supported from said pistol grip handle and a multiple linkage mechanism for supporting the actuation lever.

15. The surgical instrument of claim 1 including a tool actuation cable that extends from said tool to said handle, a slider for capturing the proximal end of said tool actuation cable and an actuation lever supported at said handle for controlling the translation of said slider.

16. The surgical instrument of claim 1 including a rotation knob supported by said control handle, said rotation knob including inner and outer rotation knob portions.

17. A medical instrument comprising a proximal control handle and a distal tool that are intercoupled by an elongated instrument shaft that is meant to pass internally of an anatomic body, proximal and distal bendable members that respectively intercouple said proximal control handle and said distal tool with said instrument shaft, cable actuation means disposed between said bendable members, for controlling the positioning of said distal tool from said control handle, and a locking mechanism for fixing the position of the tool at a selected position;
said locking mechanism comprising a ball and socket structure coupled between the control handle and proximal bendable member and a locking member for locking said ball and socket structure and having locked and unlocked states;
a compressible ring defining a partially spherical shaped socket of the ball and socket structure;
a hollow ball member of the ball and socket structure having an internal hollow chamber and an outer partially spherical shaped surface that mates with the partially spherical shaped socket;
said locking member including an annular locking ring disposed about the compressible ring, and in the locked state, compressing the socket relative to the ball member so as to fix the position of the proximal bendable member and, in turn, the distal bendable member and tool.

18. The medical instrument of claim 17 wherein said hollow ball member is supported within said partially spherical shaped socket and constructed and arranged to contact the proximal bendable member with at least a portion of the proximal bendable member disposed in the internal hollow chamber of the hollow ball member.

19. The medical instrument of claim 17 wherein said cable actuation means includes a plurality of bend control cables that are constrained along substantially the entire length thereof.

20. The medical instrument of claim 19 including a shaft filler disposed in said instrument shaft about said cables.

21. The medical instrument of claim 17 wherein the compression ring includes an annular resilient base member, and a plurality of stiffener segments that are disposed about the annular resilient base member.

22. The medical instrument of claim 21 wherein the annular resilient base member includes a plurality of spacedly disposed hinges that define therebetween a plurality of support segments.

23. The medical instrument of claim 22 wherein the plurality of stiffener segments are disposed over the respective plurality of support segments.

24. The medical instrument of claim 23 wherein each stiffener segment has, on a top surface thereof, a series of ribs.

25. The medical instrument of claim 24 wherein the ribs are spaced apart and extend circumferentially.

26. The medical instrument of claim 25 including at least a hub for supporting the compression ring.

27. The medical instrument of claim 26 including a plurality of struts for supporting the hub from the control handle.

28. The medical instrument of claim 27 wherein each hinge includes a passage for receiving a respective strut.

29. The medical instrument of claim 17 further including a rotation knob disposed adjacent the control handle and rotatable relative to the control handle for causing a corresponding rotation of the instrument shaft and tool.

30. The medical instrument of claim 17 wherein said locking member comprises a cinch ring that interlocks with a hub of the control handle.

31. The medical instrument of claim 30 wherein the cinch ring also includes a locking lever mounted from the cinch ring.

32. The medical instrument of claim 17 wherein said control handle has proximal and distal ends and including a split hub and further including a plurality of struts that connect the split hub with the distal end of the control handle.

33. The medical instrument of claim 32 wherein said split hub is annular in shape and said struts are spacedly disposed about the split hub.

34. The medical instrument of claim 33 wherein the partially spherical shaped socket is formed on an inner surface of the split hub.

35. The medical instrument of claim 34 wherein said split hub has an outer surface for receiving the locking member.

36. The medical instrument of claim 35 wherein said locking member comprises a cinch ring that interlocks with the outer surface of the split hub, and said cinch ring also includes a locking lever mounted from the cinch ring.

37. The medical instrument of claim 36 wherein the locking lever has a locked position and an unlocked position, one end of the cinch ring is in the form of a detachable hook that snap fits over a pin and sits in a slot of the lever when the ring is locked, and other end of the cinch ring is in the form of two bales that snap fit over a pin formed on the sides of the lever.

38. The medical instrument of claim 36 wherein the cinch ring is free to rotate around the split hub when the lever is released by means of a spline that rides in a groove in the outer circumference of the split hub allowing for left or right handed operation of the instrument.

39. A surgical instrument comprising:
an instrument shaft having proximal and distal ends;
a tool disposed from the distal end of the instrument shaft;
a control handle coupled from the proximal end of the instrument shaft;
a distal bendable member for coupling the distal end of said instrument shaft to said tool;
a proximal bendable member for coupling the proximal end of said instrument shaft to said control handle;
actuation means extending between said distal and proximal bendable members for coupling motion of said proximal bendable member to said distal bendable member for controlling the positioning of said tool;
and a tool actuation member that is mounted at and operable from the control handle;
said tool actuation member comprising an actuation lever extending from the control handle so as to be operable by a user, and a linkage mechanism supported at the control handle and operable from the actuation lever;
said linkage mechanism including a plurality of separate links, one of which is pivotally supported with the actuation lever.

40. The surgical instrument of claim 39 including a tool actuation cable and a slider c coupled with the tool actuation cable and controlled from the actuation lever.

41. The surgical instrument of claim 40 including a ratchet and pawl mechanism in the handle for allowing a ratcheting action by the actuation lever.

42. The surgical instrument of claim 41 including a release button on the handle coupled to the ratchet and pawl mechanism for releasing the ratchet and pawl mechanism.

43. The surgical instrument of claim 42 including a slide mechanism on the handle for dis-engaging the ratchet and pawl mechanism.

44. The surgical instrument of claim 43 including a pair of parallel disposed links supported between the actuation lever and the handle with one of the links having a ratchet surface forming part of the ratchet and pawl mechanism, a third links pivotally supported in the handle and including a distal pawl forming part of the ratchet and pawl mechanism, and a fourth link coupled between one of the pair of links and the slider.

45. The surgical instrument of claim 44 wherein the release button has a spring arm, and the release button is coupled with the third link.

46. The surgical instrument of claim 39 including a locking mechanism for fixing the position of the tool at a selected position; said locking mechanism including a ball and socket arrangement disposed about said proximal bendable member and a locking member for locking said ball and socket arrangement and having locked and unlocked states; said ball and socket arrangement including a compression ring supported from the control handle, having an outer surface for support of the locking member thereabout and having an inner surface defining an at least partially spherical shaped socket and said ball and socket arrangement further including a hollow ball member having an internal hollow chamber and an outer at least partially spherical shaped surface that mates with the at least partially spherical shaped socket.
